(19) United States
(12) Patent Application Publication
(10) Pub. No.: US 2003/0130603 A1
(43) Pub. Date: Jul. 10, 2003

(54) Orthopedic garment

(76) Inventor: Helmut W. Minne, Bad Prymont (DE)

Correspondence Address: Welsh & Katz, Ltd., THOMAS R. VIGIL, 22nd Floor, 120 South Riverside Plaza, Chicago, IL, 60606, US

(21) Appl. No.: 10/308,210

(22) Filed: Dec. 2, 2002

Related U.S. Application Data
(63) Continuation of application No. PCT/DE01/02053, filed on May 30, 2001.

(30) Foreign Application Priority Data
May 30, 2000 (DE) 20009763.6

(51) Int. Cl. A61F 5/02

(52) U.S. Cl. 602/19; 2/44

(57) ABSTRACT

The orthopedic garment is a one-piece garment especially suited for patients with osteoporosis and is made a cotton or synthetic fiber material and has different stretch zones each having a certain tension when applied. The rear area of the garment (1) is provided with a long pocket (2), which is open at the top or bottom and which accommodates a narrow rigid splint (4) that supports the vertical column of the spine. The splint is held inside the pocket (2) in a flexible manner or with a little play.

### CROSS REFERENCE TO RELATED APPLICATIONS

This application is a continuation of PCT/DE01/02053, filed May 20, 2001 and claims priority from German application no. 200 09 763.6, filed on May 30, 2000.

### BACKGROUND OF THE INVENTION

1. Field of the Invention

The present invention relates to an orthopedic garment that is particularly suited for patients with osteoporosis medical conditions. The garment of the present invention is useful to patients with osteoporosis medical conditions. Preferably, the garment is a one-piece undergarment which is made of a cotton or synthetic fiber material and which has different stretch zones, each having a certain tension when applied, and the rear area of the garment is provided with a long pocket which is open at the top or bottom and which accommodates a rigid splint that supports the spine and is held inside the pocket in one of a close-fitting manner, in a flexible manner or with a little play.

2. Description of the Prior Art

Compression fracture of a vertebral body is a typical late complication of osteoporosis. The resulting deformation of the vertebral body leads to mal-alignment of the facet articulations, to malfunction of tendons, ligaments and muscles. These cause chronical pain and disabilities in everyday-life situations.

The object of an orthosis then is to straighten the spine in order to relieve the compressed vertebral body, and with it the strained and painful periosteum, to improve the function of the facet articulations and to thus prevent permanent damage of the joints. This improves the treatment possibilities after fresh fracture and in conditions of chronic spine deformation. The present invention allows this straightening by having the kyphotic spine molded to an orthosis that is applied from the back onto the patient's back. It is thus capable of preventing pain and of improving mobilization.

All of the hereto before utilized orthoses which have this therapeutic goal try to reach it by corsetting (elastic abdominal binders) or by causing a pressure to be exerted onto structures used as abutments for straightening. This restricts the patients, impairs their mobility to a sometimes unbearable extent and results in the fact that these aids are not used to the desired extent. As a rule, this is an obstacle to an appropriate use of an actually desirable therapeutic approach.

DE-U-297 20 475 describes an osteoporosis minimal orthosis that is capable, on the one side, of desirably straightening the spine but without, due to its design, hindering chest and belly breathing and restricting the mobility in the shoulder and arm region. This has increased the patient acceptance of this new orthosis, thus impressively enhancing his treatability. Treatability of patients with osteoporosis and compression fractures of vertebral bodies is optimized.

The disadvantage thereof however is that, like most of the afore mentioned aids, they feel bulky under clothing, thus being visible or noticeable.

Further, EP-A-0 941 721 discloses a straightener with an osteoporosis orthosis in which a system of tapes and bandages perform a holding function and may fortify the muscles as well. This aid is made in two pieces and is not configured to be a garment, it does not particularly support the spine either. Bodices or corsets with supporting bones or frames (DE-U-89 04 637, DE-A-32 22 168, DE-A-32 32 638) are also known.

### BRIEF SUMMARY OF THE INVENTION

It is the object of the present invention to provide an orthopedic garment that largely appears as a “normal” garment but is very effective in treating osteoporosis by supporting the spine.

This object is achieved by providing a one-piece undergarment which is made of a cotton or synthetic fiber material and which has different stretch zones, each having a certain tension when applied, and the rear area of the garment is provided with a long pocket which is open at the top or bottom and which accommodates a rigid splint that supports the spine and is held inside the pocket in one of a close-fitting manner, in a flexible manner or with a little play.

Advantageous embodiments and developments of the invention are recited below.

In accordance with the invention, the orthopedic garment, more specifically the orthopedically indicated one-piece undergarment, especially for patients with osteoporosis medical conditions, the one-piece undergarment being made of a cotton or synthetic fiber material and having different stretch zones, each having a certain tension when applied, is characterized in that the rear area of the garment is provided with a long pocket which is open at the top or bottom and which accommodates a rigid splint that supports the spine and is held inside the pocket in a close-fitting manner, in a flexible manner or with little play.

According to a preferred embodiment of the invention, the pocket with the splint extends from the coccyx to the first cervical vertebra. The pocket closes with a zip, dome fasteners, buttons or a Velcro fastener.

According to another preferred embodiment of the invention, three stretch zones are provided, the first stretch zone extending transversely around the belly and the second and third stretch zones respectively from the chest over the shoulder to the back.

The splint is preferably thermoformable and can be adapted by the orthopedist.

According to a preferred embodiment, the garment is cut to form a one-piece undergarment closing in the crotch region (so-called body), the closure being disposed on the front, above the symphysis.

A button, zip, Velcro and/or hook and eye closure that extends downward from the front neckline may be additionally provided.

According to another embodiment of the invention, the garment is cut in the form of a one-piece cyclist outfit with legs and the closure is a button, zip, Velcro and/or hook and eye closure that extends downward from the front neckline.

In the crotch region, both embodiments of the invention may be provided with further openings for body waste excretions.

According to a particular embodiment of the invention, the splint is a flexible hollow part that is pneumatically or hydraulically stiffened, stiffening being performed externally by means of an external or internal pump or from an external or internal reservoir, valve means being provided to lower the pressure built up for stiffening.

According to another embodiment of the invention, the splint may comprise several form-fitting links, means being provided for tightening the links together, thus forming the rigid splint, as well as for loosening the rigidity thereof. The means for tightening are for example, on the one side, at least one flexible or rigid tape that connects the links together by extending through aligned bores and that is fastened at one end to an end of the splint and, on the other side, comprises a lever, eccentric lever, toggle, screwed closure or the like that is connected to the other end of the tape and is supported by the other end of the splint.

The splint is preferably provided with a recess for the spinous processes of the spine, which relieves the pressure-sensitive spinous processes. It is molded to the body of the patient beside the spinous processes. Its cross sectional diameter in the region of the trunk is increased so that the bearing area is enlarged and the pressure on each bearing area reduced.

In accordance with the invention, a splint for bandages, bodices, garments, corsets, orthoses and the like for the treatment of osteoporosis by supporting the spine, said splint extending from the coccyx to the first cervical vertebra, is characterized in that the splint is made of a thermoformable, thermoplastic material, that the splint is provided with a recess for the spinous processes of the spine and is molded to the patient's body beside said processes, that the cross sectional diameter of the splint is increased in the region of the body and that the splint is given a wave shape that conforms to the curvature of the spine. With this configuration the above mentioned advantages may also be made use of in other aids than orthopedic garments.

### BRIEF DESCRIPTION OF THE SEVERAL VIEWS OF THE DRAWINGS

The invention will be described herein after in greater detail by way of example only with reference to the drawings in which:

FIG. 1 is a back view of an orthopedic garment;

FIG. 2 is the front view of the garment of FIG. 1;

FIG. 3 is a perspective illustration of a splint for the garment of FIGS. 1 and 2.

### DETAILED DECRIPTION OF THE INVENTION

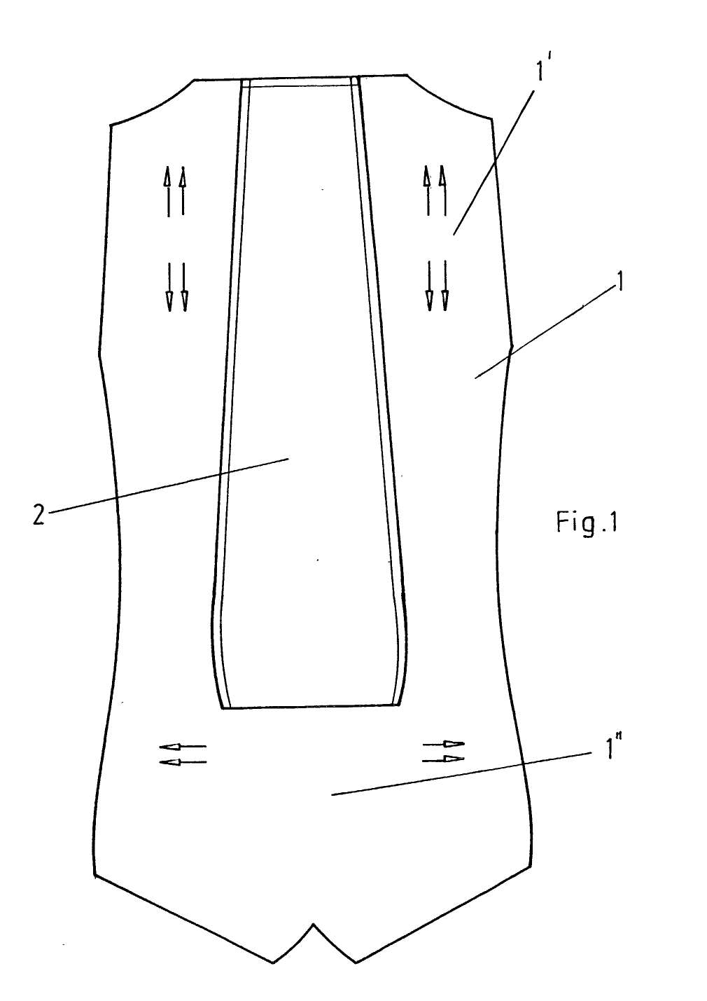

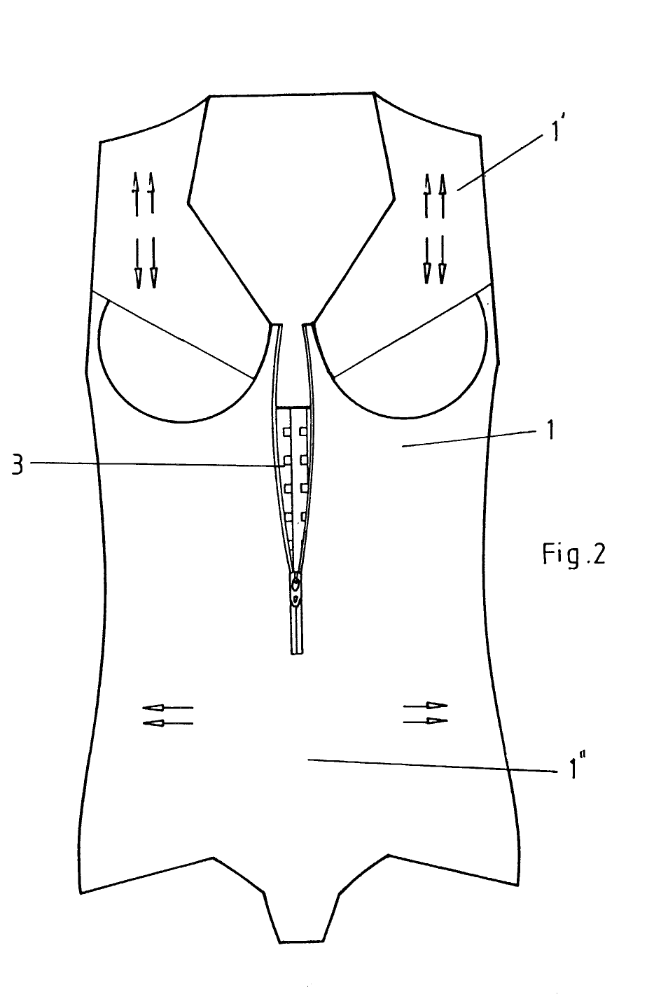

The orthopedic garment 1 according to the FIGS. 1 and 2, which is cut like a conventional body and is shown schematically, is provided on its rear area with an appliqued insert pocket 2 that extends along the spine, is closed on the sides and at the top but open at the bottom and receives the orthopedic splint that supports the spine from the coccyx to the first cervical vertebra. The pocket 2 conforms to the shape of the splint and is accordingly wider at the bottom, tapering toward the neck. The splint is removably fastened inside the pocket by means of a Velcro fastener. The open side of the pocket may also close with a zip, dome fasteners or the like. To the front, the garment 1 has a zip 3 extending from the neckline. The garment 1 is further provided with three stretch zones made from woven elastic fabric; several layers of fabric may be superimposed for stiffening. One stretch zone 1″ extends horizontally around the abdomen whereas the two other stretch zones 1′ each extend vertically over the shoulders at the front and at the back.

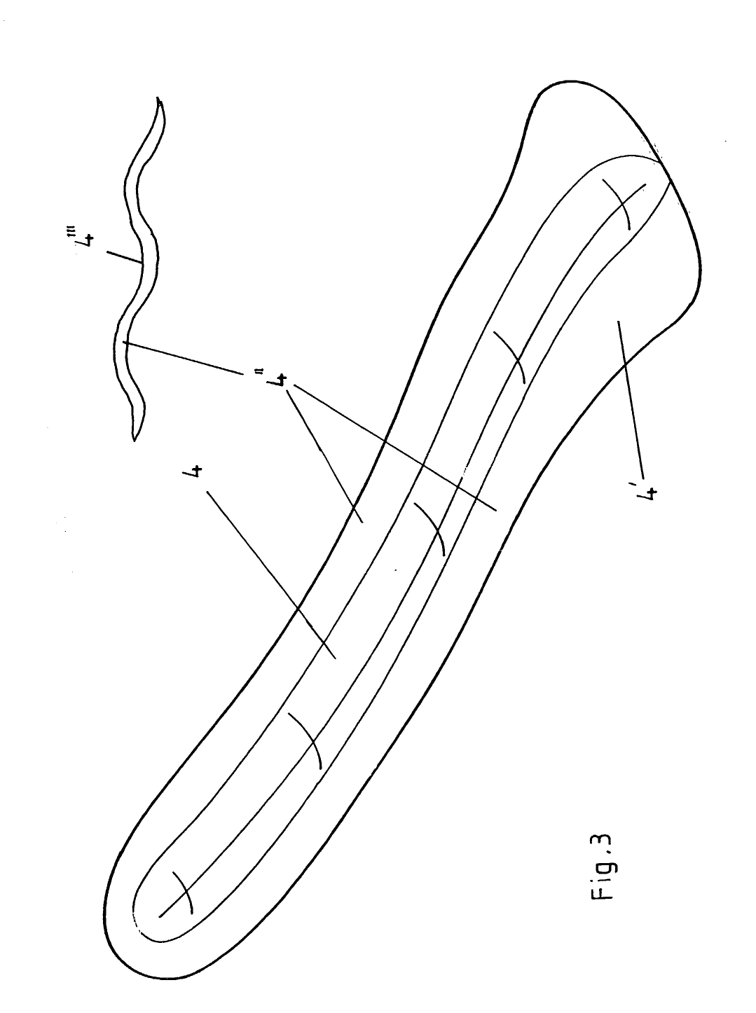

The splint 4 shown in FIG. 3 that is to be introduced into the insert pocket on the rear area of the orthopedic garment has a slightly curved contour that conforms to the shape of the spine and gets wider at the lower end 4′ thereof. On the sides thereof, broadenings 4″ are provided that are brought to fit the body of the patient, as may be surveyed from the cross sectional view represented on the right side of the Fig., a recess 4′″ that straddles the spinous processes of the spine being provided between said broadenings. In practical use, such a pre-fabricated but thermoformable splint may be molded to become very flat and snugly fit the spine of the patient. Thanks to the wave shape of the recess 4′″, the inward curved broadenings 4′ and their terminating, slightly outward curved ends, as shown in the cross sectional view, said splint is mechanically very stable so that a flatter, molded splint has the same stability than a pre-fabricated splint that has not been molded to the body.

1. An orthopedic garment (1), more specifically an orthopedically indicated one-piece undergarment, especially for patients with osteoporosis medical conditions, the one-piece undergarment being made of a cotton or synthetic fiber material and having different stretch zones, each having a certain tension when applied,
characterized in that
the rear area of the garment (1) is provided with a long pocket (2) which is open at the top or bottom and which accommodates a rigid splint (4) that supports the spine and is held inside the pocket in a close-fitting manner, in a flexible manner or with little play.

2. The orthopedic garment according to claim 1,
characterized in that
the pocket (2) with the splint (4) extends from the coccyx to the first cervical vertebra.

3. The orthopedic garment according to claim 1, characterized in that the pocket (2) closes with a zip, dome fasteners, buttons or a Velcro fastener.

4. The orthopedic garment according to claim 1,
characterized in that
there are three stretch zones (1′, 1″), the first stretch zone extending transversely around the belly and the second and third stretch zones respectively from the chest over the shoulder to the back

5. The orthopedic garment according to claim 1,
characterized in that
the splint (4) is thermoformable.

6. The orthopedic garment according to claim 1,
characterized in that
the garment (1) is cut to form a one-piece undergarment closing in the crotch region (so-called body).

7. The orthopedic garment according to claim 6,
characterized in that
the closure is disposed on the front, above the symphysis.

8. The orthopedic garment according to claim 6,
characterized in that
a button, zip, Velcro and/or hook and eye closure that extends downward from the front neckline is additionally provided.

9. The orthopedic garment according to claim 1,
characterized in that
the garment is cut in the form of a one-piece cyclist outfit with legs and that the closure is a button, zip, Velcro and/or hook and eye closure that extends downward from the front neckline.

10. The orthopedic garment according to claim 6,
characterized in that
further openings for body waste excretions are provided in the crotch region.

11. The orthopedic garment according to claim 1,
characterized in that
the splint is a flexible hollow part that is pneumatically or hydraulically stiffened.

12. The orthopedic garment according to claim 11,
characterized in that
stiffening is performed externally by means of an external or internal pump or from an external or internal reservoir, valve means being provided to lower the pressure built up for stiffening.

13. The orthopedic garment according to claim 1,
characterized in that
the splint comprises several form-fitting links, means being provided for tightening the links together, thus forming the rigid splint, as well as for loosening the rigidity thereof.

14. The orthopedic garment according to claim 13,
characterized in that
the means for tightening are, on the one side, at least one flexible or rigid tape that connects the links together by extending through aligned bores and that is fastened at one end to an end of the splint, and, that, on the other side, the means for tightening comprises a lever, eccentric lever, toggle, screwed closure or the like that is connected to the other end of the tape and is supported by the other end of the splint.

15. The orthopedic garment according to claim 1,
characterized in that
the splint is provided with a recess for the spinous processes of the spine and is molded beside them, to the left and right side thereof, to the body of the patient.

16. The orthopedic garment according to claim 15,
characterized in that,
in the region of the trunk, the cross sectional diameter of the splint is increased.

17. A splint for bandages, bodices, garments, corsets, orthoses and the like for the treatment of osteoporosis by supporting the spine, said splint (4) extending from the coccyx to the first cervical vertebra,
characterized in that
the splint is made of a thermoformable, thermoplastic material,
that the splint is provided with a recess (4′″) for the spinous processes of the spine and is molded to the patient's body beside said processes,
that the cross sectional diameter (4′) of the splint is increased in the region of the trunk, and,
that the splint is given a wave shape that conforms to the curvature of the spine.
